(19) United States
(12) Patent Application Publication
(10) Pub. No.: US 2006/0293737 A1
(43) Pub. Date: Dec. 28, 2006

(54) Multiple electrode implantable lead

(75) Inventor: Mohan Krishnan, Shoreview, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/158,745

(22) Filed: Jun. 22, 2005

(51) Int. Cl. A61N 1/05

(52) U.S. Cl. 607/122

(57) ABSTRACT

This document discusses, among other things, a lead assembly including a lead body, a first conductor extending through the lead body and coupled to a first electrode, a second conductor extending through the lead body and coupled to a second electrode, and a multi-filar coil extending through the lead body. The multi-filar coil includes electrically independent first and second filars respectively coupled to first and second sensing or pacing electrodes. In an example, the second filar of the multi-filar coil is substantially coaxial with the first filar. An example method includes extending first and second conductors and a multi-filar coil through lumens in a lead body and coupling electrodes to the conductors and coils.

### TECHNICAL FIELD

This patent document pertains generally to medical device lead assemblies, and more particularly, but not by way of limitation, to a multiple electrode implantable lead.

### BACKGROUND

Medical device lead assemblies typically include a lead body and at least one conductor extending through the lead body. Lead assemblies are often used in conjunction with an implantable medical device, such as a pacer and defibrillation, or neural stimulator.

An example pacer lead assembly includes a pacing electrode, an insulative lead body, and a conductor that extends through the lead body and is electrically coupled to the pacing electrode. In some examples, the pacing electrode is near a distal end of the lead assembly, and a proximal end of the lead assembly includes a connector that couples the lead assembly to a medical device.

Defibrillation leads includes at least one defibrillation electrode, such as a defibrillation coil. An example defibrillation lead assembly includes two defibrillation electrodes, two conductors that extend through the lead body and couple to the respective defibrillation electrodes, and a connector assembly that couples the defibrillation electrodes to a medical device, which typically includes a pulse generator. Some defibrillation lead assemblies also include pacing and/or sensing electrodes and additional conductors that couple to the pacing and sensing electrodes to the medical device.

Other types of lead assemblies also include multiple conductors and multiple electrodes. Improved lead assemblies are needed.

### SUMMARY

An example lead assembly includes a lead body, a first conductor extending through the lead body and coupled to a first electrode, a second conductor extending through the lead body and coupled to a second electrode, and a multi-filar coil extending through the lead body. The multi-filar coil includes a first filar coupled to a first sensing or pacing electrode, and a second filar coupled to a second sensing or pacing electrode, the first filar electrically independent from the second filar.

Another example medical device includes a pulse generator, a connector block, a lead assembly coupleable to the connector block. The lead assembly includes a lead body, a first conductor extending through the lead body and coupled to a first electrode, a second conductor extending through the lead body and coupled to a second electrode, and a multi-filar coil including a plurality of filars extending through the lead body. The multi-filar coil includes a first filar coupled to a first sensing or pacing electrode and a second filar coupled to a second sensing or pacing electrode.

A example method includes extending a first conductor through a lead body including at least one lumen, extending a second conductor through the lead body, extending a multi-filar coil through the lead body, the multi-filar coil including at least a first filar and a second filar, coupling a first electrode to the first conductor, coupling a second electrode to the second conductor, coupling the first filar of the multi-filar coil to a first sensing or pacing electrode and coupling the second filar of the multi-filar coil to a second sensing or pacing electrode.

### BRIEF DESCRIPTION OF THE DRAWINGS

FIGS. 1A and 1B are illustrations of a lead assembly and a heart.

FIG. 2 is an illustration of a lead assembly including multiple electrodes.

FIG. 3A is a partially cut-away illustration of an example lead assembly including a multi-filar coil.

FIG. 3B is a cross-section of the lead assembly of FIG. 3.

FIG. 3C is a partially cut-away illustration of another example lead assembly including a quad-filar coil.

FIG. 4 is a flow chart that illustrates an example method.

FIG. 5 is a cross-sectional illustration of a distal portion of an example lead assembly.

### DETAILED DESCRIPTION

The following detailed description includes references to the accompanying drawings, which form a part of the detailed description. The drawings show, by way of illustration, specific embodiments in which the invention may be practiced. These embodiments are also referred to herein as “examples.” The drawings and following detailed description is not to be taken in a limiting sense, and the scope of the present invention is defined by the appended claims and their equivalents.

A medical device lead assembly includes a multi-filar coil that includes at least two electrically independent filars. In an example, the multi-filar coil includes two filars, at least one of which includes a coating of insulation. The filars extend along a helical path and form the multi-filar coil. The helical path optionally has a constant radius, and/or a constant pitch. The filars of the multi-filar coil are optionally coradial. In an example, each filar of the coil is insulated. In an example, the multi-filar coil extends through a lumen in a multi-lumen lead body. Cables or other conductors extend through other lumens in the lead body. The use of a multi-filar coil facilitates fabrication of small-diameter leads and/or multi-conductor leads, including leads that have four or more conductors, for example. Reducing the size of leads can be desirable, for example to avoid interference with heart valve functions.

A multi-filar coil allows for efficient use of space in a lead body. For example, a multi-filar coil typically takes up about the same amount of cross-sectional space as a single-filar coil of the same size, while providing two or more electrical connections instead of one. In contrast, in a lead assembly that does not include a multi-filar coil, an increase in the number of conductors is usually accompanied by an increase in the overall size (e.g. diameter) of the lead assembly, a reduction in the diameter of one or more conductors, or a reduction in tubing wall thickness. Reducing the conductor diameter can be problematic, for example, because some small-diameter conductors can be difficult to string through a lumen in a lead body. Reducing wall thickness of tubing can affect abrasion performance.

A multi-filar coil can also be used to achieve desirable handling characteristics. Lead assemblies with particularly low bending stiffness can be difficult to handle. For example, it is difficult to push a highly flexible lead through a blood vessel, because the lead bends when it is urged through the vessel. Increasing the bending stiffness of a lead assembly can improve the handling or “pushability” of a lead. A coiled filar tends to be stiffer than a straight filar of the same wire diameter. In an example, a multi-filar coil provides increased stiffness and desirable handling or pushability characteristics. In an example, the size and number of filars in a coil are adjusted to adjust the handling characteristics. In another example, a multi-filar coil that is coupled to pacing or sensing conductors is combined with high-voltage conductors such as defibrillation cables to provide a tachycardia lead with desirable size and bending characteristics.

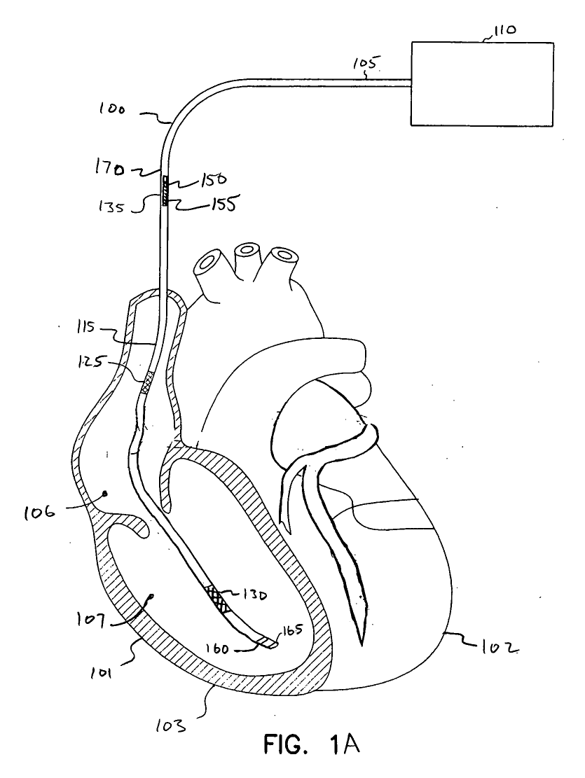

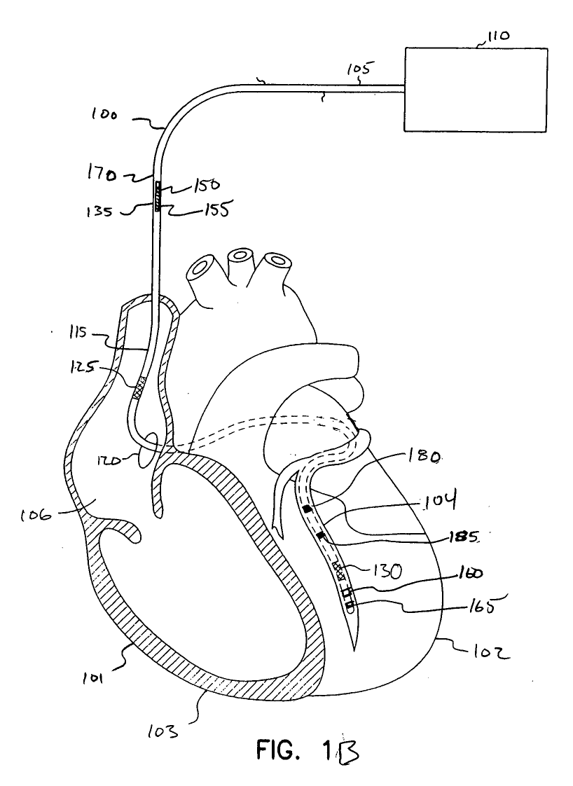

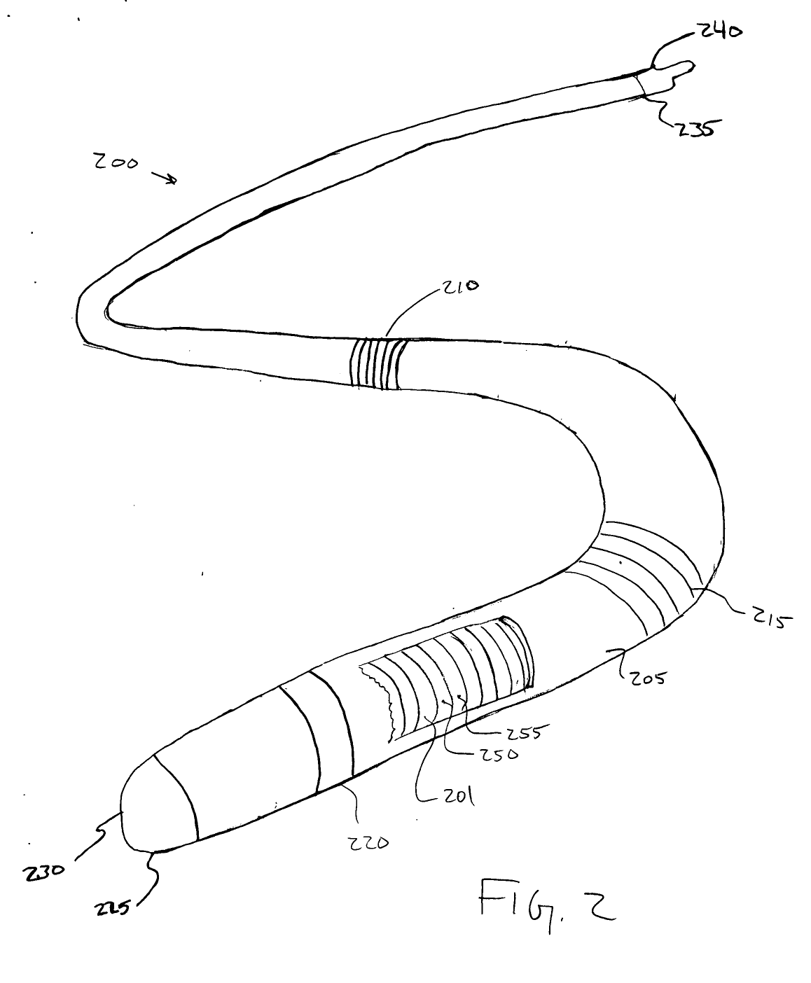

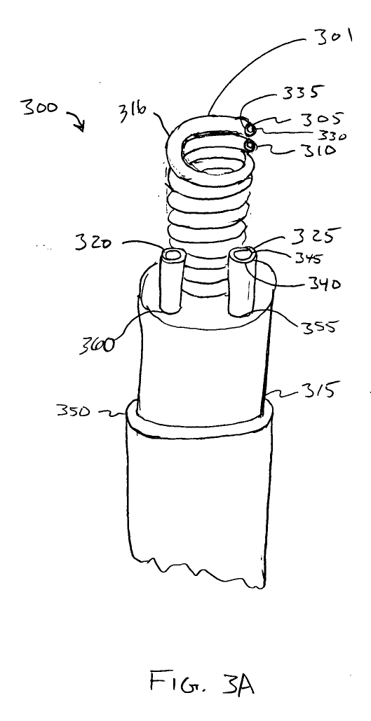

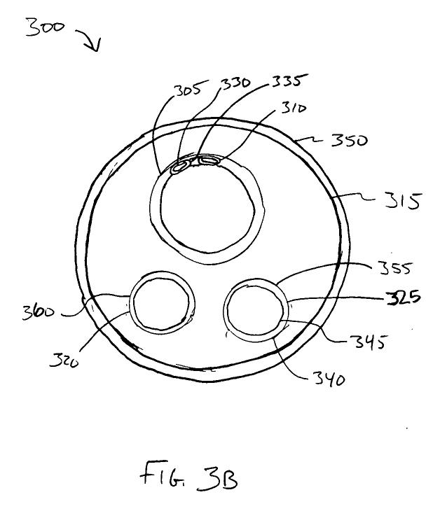

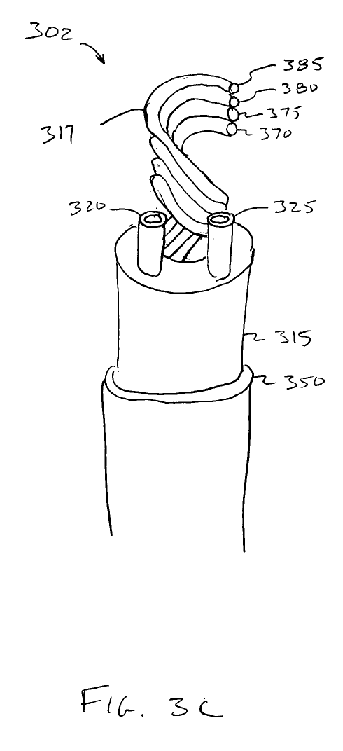

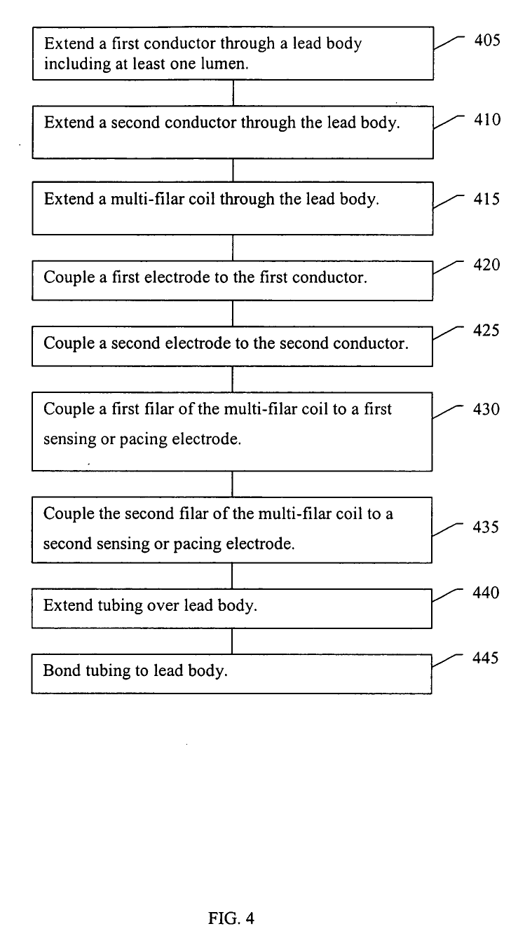

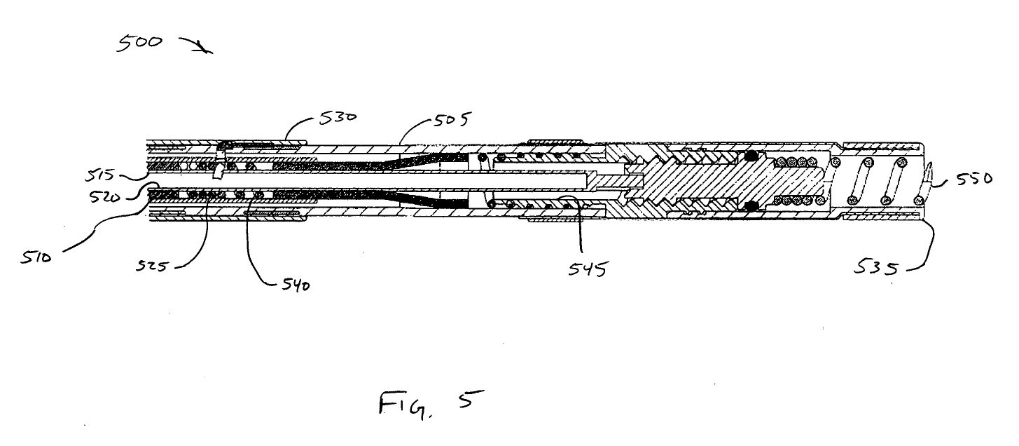

FIGS. 1A and 1B show examples of lead assemblies extending from a pulse generator into a heart. The lead assembly includes a lead body and a multi-filar coil including two or more electrically isolated filars. FIG. 2 shows another example lead assembly having multiple electrodes and a multi-filar coil. FIG. 3A shows a partially cutaway view of an example lead assembly that includes a multi-filar coil and two additional conductors. A cross-section of the example lead assembly shown in FIG. 3A is provided in FIG. 3B. While a multi-filar coil having two filars is shown in FIG. 3A, it is understood that a multi-filar coil can include three, four, or more filars. An example lead assembly having a quad-filar coil is shown in FIG. 3C. FIG. 4 is a flow chart that illustrates an example method. FIG. 5 shows a cross-section of a distal portion of an example lead assembly including electrodes coupled to filars of a multi-filar coil. While a multi-filar coil having two filars is shown in FIG. 3A, it is understood that a multi-filar coil can include three, four, or more filars.

Turning now to FIGS. 1A and 1B, an example lead assembly 100 includes a proximal portion 105 that is coupled to a pulse generator 110, and a distal portion 115 that extends in, on, or around a heart 101. The lead assembly 100 includes a lead body 170 and a multi-filar coil 135 including two or more filars 150, 155 extending through a lumen in the lead body. In an example, the filars are 150, 155 are coupled to respective sensing or pacing electrodes 160, 165. In an example, the sensing or pacing electrodes 160, 165 are usable for sensing an intrinsic electrical heart signal and/or pacing a heart. In an example, electrode 165 includes an active fixation helix. In an example, the lead assembly 100 also includes two or more defibrillation 125, 130 electrodes. In an example, the defibrillation electrodes 125, 130, each include a defibrillation coil that is coupled to a high-voltage cable extending through the lead body 170.

In the example shown in FIG. 1A, the lead assembly 100 extends through the superior vena cava (SVC) into the right atrium 106 and right ventricle 107. In another alternative example, the lead assembly 100 extends on or around the right side 103 of the heart 101. In other examples, the lead assembly 100 extends on or around the heart, or in, on, or around another location in the body.

In the example shown in FIG. B, the lead assembly 100 extends through the coronary sinus 120 to the left side 102 of the heart 101. In an example, the lead assembly extends through a vessel 104 on the left side of the heart. In an example, the lead assembly 100 is a multi-polar lead. In the example shown in FIG. 1B, the lead assembly 100 includes third and fourth sensing or pacing electrodes 180, 185. In an example, the multi-filar coil 135 includes third and fourth filars that are respectively coupled to the third and fourth sensing or pacing electrodes 180, 185. In an example, a connector on the lead assembly includes sufficient electrical contacts to simultaneously connect all four sensing or pacing electrodes 160, 165, 180, 185 to the pulse generator. In another example, switching capability is provided in the lead assembly 100, and a subset (e.g. two) of the electrodes can be simultaneously electrically connected to pulse generating or analysis circuitry in the pulse generator.

Referring now to FIG. 2, an illustration of an example lead assembly is shown. The lead assembly 200 includes a lead body 205, defibrillation electrodes 210, 215, and pacing or sensing electrodes 220, 225. The pacing or sensing electrodes 220, 225 may be used for pacing, sensing, or both. In an example, electrode 220 is a ring electrode, and electrode 225 includes a fixation helix. In an example, the electrodes 220, 225 include platinum or titanium coated with a combination of iridium oxide (IrOx), titanium/nickel (Ti/Ni), black platinum (Pt black) or tantalum oxide. The lead assembly 200 also includes a multi-filar coil 201 that includes at least two filars 250, 255. The lead body 205 is shown partially cut-away in FIG. 2 to show the filars 250, 255 of the multi-filar coil 201 extending through the lead body 205. In an example, the filars 250, 255 are approximately coradial. In an example, the pacing or sensing electrodes 220, 225 are located near a distal end portion 230 of the lead assembly 200. In an alternative example, the pacing or sensing electrodes are located elsewhere on the lead assembly 200. In an example, a proximal end portion 235 of the lead assembly coupled is to a pacer, defibrillator, stimulator, or other medical device. In an example, the lead assembly includes a connector 240 at the proximal end 235 of the lead assembly that is sized and shaped to interface with a connector block or other component of a medical device. In an example, the connector is a modified IS-4 terminal that is sized and shaped to couple with the multi-filar coil.

Turning now to FIGS. 3A and 3B, an example lead assembly 300 has a multi-filar coil that includes two filars. FIG. 3A is a partially cut-away illustration of an example lead assembly 300. FIG. 3B is a cross-section of the lead assembly 300. The lead assembly 300 includes a lead body 315 and multi-filar coil 301 extending through a lumen 316 in the lead body. In an example, the multi-filar coil 301 includes two filars 305, 310. In an example, the filars 305, 310 are approximately coradial, i.e. the filars follow helical paths having approximately the same radius and approximately the same axis. In an example, at least a portion of the helical paths followed by the filars 305, 310 have a constant radius and a constant pitch.

Referring again to FIGS. 3A and 3B, the lead assembly 300 also includes one or more additional conductors. In the example shown in FIGS. 3A and 3B, the lead assembly 300 includes two additional conductors 320, 325 that extend through second and third lumens 355, 360 in the lead body 315. In an example, the conductors 320, 325 are approximately equally spaced from each other and from the multi-filar coil. In an example, the conductors 320, 325 are cabled from small diameter wire.

In an example, the lead assembly 300 is configured as shown in FIG. 1A or FIG. 2, i.e. the lead assembly has two pacing/sensing electrodes and two defibrillation electrodes. In an example, the coils 305, 310 are respectively coupled to the pacing/sensing electrodes, and conductors 320, 325 are each coupled to a defibrillation electrode. In an example, one or both of the conductors 320, 325 is a cable that includes a plurality or multiplicity of filars.

Referring now to FIG. 3C, another example lead assembly 302 includes a multi-filar coil 317 including four filars 370, 375, 380, 385. Filar 370 contacts filar 375, filar 375 contacts 380, and filar 380 contacts filar 385. There is a gap between filar 385 and filar 370. In another option, filar 370 contacts filar 385 and the filars define a close tube. At least two of the filars 370, 375, 380, 385 are electrically independent. At least one filar includes a coating of insulation. In an example, all four filars 370, 375, 380, 385 are electrically independent. In another option, two or more filars are electrically connected. In an example, filar 370 is electrically connected with filar 375 and filar 380 is electrically connected to filar 385. In an example, the lead assembly 302 is configured as shown in FIG. 1B, i.e. the lead assembly has two defibrillation electrodes respectively coupled to conductors 320, 325 and four sensing or pacing electrodes respectively coupled to filars 370, 375, 380, 385. In another example, one or more filars is not used as a conductor, and is provided to facilitate control of bending and stiffness properties of the lead assembly.

Referring again to FIGS. 3A-C, in an example, the lead body 315 includes silicone rubber, polyurethane elastomer, or a fluoropolymer. In an example, one or more of the filars in the multi-filar coil includes a conductive core 330 and an insulative cover 335. In an example, the conductive core 330 is an alloy such as MP35N with a silver core. In another example, the conductor is platinum-clad tantalum (Pt/Ta), or platinum-clad tantalum with a silver core. In an example, conductors 320, 325 also include an insulative outer layer 340 and a conductive core 345. In an example, the conductive core 345 includes stainless steel, MP35N with a silver core, platinum-clad tantalum, or platinum-clad tantalum with a silver core. In an example, one or both of the conductors 320, 325 include drawn brazed strand (DBS®) cable.

In an example, the electrodes include platinum or titanium coated with IrOx, titanium/nickel (Ti/Ni), black platinum (Pt black) or tantalum oxide. In an example, the lead assembly also includes an outer covering 350 that extends over the lead body 315. In an example, the outer covering includes ethylene-tetrafluoroethylene (ETFE), polytetrafluoroethylene (PTFE), polyethylene (PE), silicone rubber, or polyurethane. In an example, the lead assembly 300 shown in FIGS. 3A and 3B is used to deliver cardiac resynchronization therapy (CRT), neural stimulation, antibradyarrhythmia therapy (e.g. pacing) or antitachyarrhythmia therapy (e.g. defibrillation).

Referring now to FIG. 4, an example method of making a lead assembly including a multi-filar coil is schematically illustrated in a flowchart. At 405, a first conductor is extended through lead body including at least one lumen. At 410, a second conductor is extended through the lead body. In an example, the first conductor and/or the second conductor include a multi-wire cable. In an example, the first and second conductors are extended through separate lumens. At 415, a multi-filar coil is extended through the lead body. The multi-filar coil includes two or more electrically independent coiled filars. In an example, the filars of the multi-filar coil are coradial. In an example, the filars are wound on a mandrel into a coil before the multi-filar coil is extended through the lead body. In an example, the first and second conductors are extended through respective first and second lumens, and the multi-filar coil is extended through a third lumen. At 420, a first electrode is coupled to the first conductor. At 425, a second electrode is coupled to the second conductor. At 430, the filar of the multi-filar coil is coupled to a first sensing or pacing electrode. In an example, the first sensing or pacing electrode is configured for use in both sensing and pacing operations. At 435, a second filar of the multi-filar coil is coupled to a second sensing or pacing electrode. At 440, tubing is extended over the lead body. In an example, the tubing makes part or all of the lead body isodiametric. At 445, the tubing is optionally bonded to the lead body. In an example, the tubing is formed from the same material as the lead body. In an example, the tubing and lead body are formed from silicone or polyurethane. In an example, the tubing is fused to the lead body using heat fusion or laser fusion, for example. In an example, the resulting product is a lead assembly including a lead body, conductors and a multi-filar coil extending through the lead body, and tubing extending over and connected to the lead body. In an example, the resulting lead assembly is isodiametric.

In an example, the operations illustrated in FIG. 4 are performed in order starting at the top with FIG. 4 and progressing downward through 440 or 450. Alternatively, the operations are performed in a different order. For example, in one option, the multi-filar coil is extended through the lead body before the conductors are extended through the lead body. In an example, fewer than all of the operations are performed. For example, in one option, the extending tubing over the lead body (at 445) and the bonding of the tubing to the lead body (at 450) are omitted, and the result of the method is a lead body including conductors and a multi-filar coil extending through the lumens.

Referring now to FIG. 5, a cross-sectional illustration of a distal portion of a lead assembly 500 is shown. The lead assembly 500 includes a lead body 505 and a multi-filar coil 510 extending through the lead body 505. In an example, a driver component 515 with an optional lumen 520 extends through the multi-filar coil 510. In an example, one filar 525 of the multi-filar coil 510 couples to an electrode 530 which is optionally near a distal end 535 of the lead assembly 500. A second filar 540 extends past a location where the first filar terminates and couples to a component 545 that is electrically coupled to an active fixation helix 550.

The above specification, examples and data provide a complete description of the manufacture and use of the composition of the invention. Since many embodiments of the invention can be made without departing from the scope of the invention, the invention resides in the claims hereinafter appended.

What is claimed is:

1. A lead assembly comprising:
a lead body;
a first conductor extending through the lead body and coupled to a first electrode;
a second conductor extending through the lead body and coupled to a second electrode;
a multi-filar coil extending through the lead body, the multi-filar coil including at least a first filar coupled to a first sensing or pacing electrode, and a second filar coupled to a second sensing or pacing electrode, the first filar electrically independent from the second filar.

2. The lead assembly of claim 1, wherein the first filar is substantially coradial with the second filar.

3. The lead assembly of claim 1, wherein the first filar includes a wire and coating of insulation over the wire.

4. The lead assembly of claim 1, wherein the lead body includes a first lumen, a second lumen, and a third lumen, the first conductor extending through the first lumen, the second conductor extending through the second lumen, and the multi-filar coil extending through the third lumen.

5. The lead assembly of claim 1, wherein the first electrode and second electrode are defibrillation electrodes.

6. The lead assembly of claim 1, wherein an antitachyarrhythmia therapy is deliverable through the first and second electrodes.

7. The lead assembly of claim 1, wherein at least one of the first conductor and the second conductor includes a cable including a plurality of wires.

8. The lead assembly of claim 1, wherein the multi-filar coil further comprises a third filar.

9. The lead assembly of claim 8, wherein the multi-filar coil further comprises a fourth filar.

10. The lead assembly of claim 9, wherein the third filar is electrically independent from the first filar and the second filar and is coupled to a third electrode, and the fourth filar is electrically independent from the first, second, and third filars and is coupled to a fourth electrode.

11. The lead assembly of claim 1, wherein at least one of the first sensing or pacing electrode and the second sensing or pacing electrode is adapted for both sensing and pacing.

12. A medical device comprising:
a pulse generator;
a connector block; and
a lead assembly coupleable to the connector block, the lead assembly including
a lead body;
a first conductor extending through the lead body and coupled to a first electrode;
a second conductor extending through the lead body and coupled to a second electrode;
a multi-filar coil including a plurality of filars extending through the lead body, the multi-filar coil including a first filar coupled to a first sensing or pacing electrode and a second filar coupled to a second sensing or pacing electrode.

13. The medical device of claim 12, wherein the multi-filar coil further comprises at least one additional filar.

14. The medical device of claim 12, wherein an antitachyarrhythmia therapy is deliverable from the pulse generator through the first and second electrodes.

15. The medical device of claim 12, wherein the first conductor includes a first cable and the second conductor includes a second cable.

16. A method comprising:
extending a first conductor through a lead body including at least one lumen;
extending a second conductor through the lead body;
extending a multi-filar coil through the lead body, the multi-filar coil including at least a first filar and a second filar;
coupling a first electrode to the first conductor;
coupling a second electrode to the second conductor;
coupling the first filar of the multi-filar coil to a first sensing or pacing electrode; and
coupling the second filar of the multi-filar coil to a second sensing or pacing electrode.

17. The method of claim 16, wherein extending a first conductor through a lead body includes extending a cable through the at least one lumen in the lead body.

18. The method of claim 16, wherein extending a first conductor through a lead body includes extending the first conductor through a first lumen in the lead body, and extending a second conductor through the lead body includes extending the second conductor through a second lumen in the lead body.

19. The method of claim 16, wherein extending a multi-filar coil through the lead body includes extending the multi-filar coil through a third lumen in the lead body.

20. The method of claim 16, further comprising winding the first filar and second filar around a mandrel and forming the multi-filar coil on the mandrel.
